(19) United States
(12) Patent Application Publication
(10) Pub. No.: US 2014/0005481 A1
(43) Pub. Date: Jan. 2, 2014

(54) METHOD AND APPARATUS FOR COLD PLASMA TREATMENT OF INTERNAL ORGANS

(71) Applicants: Michael Rontal, Farmington Hills, MI (US); David Ruzic, Pesotum, IL (US); Brian Jurczyk, Champaign, IL (US); Robert Stubbers, Champaign, IL (US)

(72) Inventors: Michael Rontal, Farmington Hills, MI (US); David Ruzic, Pesotum, IL (US); Brian Jurczyk, Champaign, IL (US); Robert Stubbers, Champaign, IL (US)

(21) Appl. No.: 13/963,364

(22) Filed: Aug. 9, 2013

Related U.S. Application Data
(63) Continuation of application No. 13/595,378, filed on Aug. 27, 2012.
(60) Provisional application No. 61/527,289, filed on Aug. 25, 2011.
(60) Provisional application No. 61/550,973, filed on Oct. 25, 2011.
(60) Provisional application No. 61/561,491, filed on Nov. 18, 2011.
(60) Provisional application No. 61/567,165, filed on Dec. 6, 2011.

(51) Int. Cl. A61B 18/04

(52) U.S. Cl. CPC A61B 18/042
USPC 600/158; 604/26; 604/20

(57) ABSTRACT

Chronic sinusitis is treated by the application of cold plasma or plasma-activated species to the infected mucosal surfaces through use of an endoscope having a steerable end which may be projected into the sinus cavities through the nasal cavity. The cold plasma is generated at either the distal end of the endoscope with a power source by application of a power, or at the distal end by gas and electrical connections extending through the endoscope. The cold plasma or plasma-activated species act to destroy bacterial cells but not eukaryotic cells.

### CROSS-REFERENCE TO RELATED APPLICATIONS

This application is a Continuation of application Ser. No. 13/595,378 filed on Aug. 27, 2012. Application Ser. No. 13/595,378 claims the benefit of U.S. Provisional Application 61/527,289 filed on Aug. 25, 2011. Application Ser. No. 13/595,378 claims the benefit of U.S. Provisional Application 61/550,973 filed on Oct. 25, 2011. Application Ser. No. 13/595,378 claims the benefit of U.S. Provisional Application 61/561,491 filed on Nov. 18, 2011. Application Ser. No. 13/595,378 claims the benefit of U.S. Provisional Application 61/567,165 filed on Dec. 6, 2011.

### FIELD OF THE INVENTION

This invention relates to methods and apparatus for treating internal organs with non-thermal plasma or plasma-activated species and more particularly to an endoscope specifically designed to apply non-thermal plasma to the mucosal lining of the paranasal sinuses and the walls of other body cavities to destroy bacteria and biofilm causing surface and invasive infections.

### BACKGROUND OF THE INVENTION

Sinusitis in its many presentations is a common medical diagnosis affecting between 35 and 40 million patients each year in the United States. It accounts for over $3 billion dollars in medication treatment alone. According to the Bureau of Labor Statistics (2010) sinusitis costs over $8.6 billion in lost productivity.

Sinusitis represents an infectious and inflammatory response of the mucous membrane coverings of the paranasal sinuses. The paranasal sinuses are spaces within the facial and skull base bones that have developed as expansions from the nasal cavity. They have a tiny opening between the sinus and the nasal cavity. The space may be upwards of 3 centimeters anterior to posterior and 1.5 to 2.0 centimeters from nasal septum to medial orbital wall. The lining of the sinuses is very much like the lining of the nose. The sinuses lie in the very narrow space between the nasal cavity, the eye and the brain with the intervening bone being very thin.

The covering of these spaces is a continuation of the nasal mucosa The sinus mucosa consists of surface or epithelial cells with microscopic projections on its surface, the cilia. The mucosal surface contains tiny glands that produce mucous, a mucopolysaccharide material that carries in it antibacterial enzymes, e.g., lysozyme and the specific immunoglobulin IgA. It is the nature of the mucous that bacteria and fungi stick to it and that the cilia beat in a fashion that forcefully moves this material with captured bacteria and fungi to the sinus opening and into the nasal cavity. This mucous is then slid to the throat where it is automatically swallowed and destroyed. The mucosal cilia beat in a coordinated fashion and can very forcefully move the mucous on the surface. The sinus cavity is thus kept sterile. The mucosa with a mucous layer and functioning cilia is a major cleaning and protection of the mucosa and eventually the respiratory tract. Treatment of the sinuses must preserve the cilia.

Sinusitis is the result of invasion and destruction of epithelial cells by viruses, bacteria, fungi, and biofilms (“pathogens”) that have been able to move deep to the mucous layer. There is a resultant inflammatory, protective reaction in the submucosa. Some of these inflammatory cells are also found on the mucosal surface. There are first responder cells during an early or acute phase infection and later inflammatory cells associated with a long-standing or chronic phase of infection.

Present day treatment of sinusitis is based on a century old concept that the cavity is filled with infected mucous and if the sinus cavity is surgically opened allowing pus to drain out and air to enter the mucosa will heal itself. In acute sinusitis this is usually the case. In chronic sinusitis where most of today's surgical approaches are aimed, there is usually little pus and the mucosa is edematous to the point of obliterating the sinus space. Unfortunately, while the immediate clearance of the stuffiness and drainage from sinusitis is made better, the long-term outlook is for recurrent infections with its drainage and stuffiness.

In 2002, in a paper in the Journal of the American Medical Association, it was shown that biofilm is associated with particularly difficult to eradicate, culture “negative” infections of the middle ear. Subsequently in 2004-2005 one of the present inventors and several others in the field showed that on the similar mucosal surfaces in the paranasal sinuses also involved in chronic infection, that there was bacterial and fungal biofilm resident on the surface. It has been shown that biofilm is present in up to 90% of chronic infected tissue samples. Whether the biofilm is causative of the initial persistence of infection or its presence is a continuing factor to keep an infection active is not known. Intuitively, however, its overwhelming presence has to make it a prime target for treatment.

Biofilms consist of bacteria and/or fungi imbedded within a self produced polysaccharide matrix. This matrix has channels within that allow the inflow of nutrients and oxygen and the removal of waste materials. It is the products of bacterial metabolism that destroy epithelial cells and initiate host immune attack, e.g. such as the Staphylococcal superantigen. The channels are too small to admit inflammatory immune mediated white blood cells that would directly attack the living organisms. The channels also limit and may be active in preventing antibiotics from accessing the bacteria or fungi. Biofilms exist at low metabolic rates and thus many antibiotics which disrupt cell metabolic activity have little substrate to effect. The biofilm may have different species of bacteria. These biofilm bacteria “talk” to each other by chemicals that signal other bacteria by up-regulating or down-regulating bacterial DNA. There is even a transfer of specific DNA code information that allows persisting bacteria to increase the resistance of other bacteria in the biofilm and thus improving the survival of the entire colony. Roughly every 9-12 days these biofilms break up to spew out pioneer cells that establish new colonies. The presence of biofilm has been identified with a more serious and recalcitrant infection.

To date there is no proof that biofilm is the cause of chronic sinusitis: however, what is shown is that the real action in the pathophysiology of sinusitis is on the surface and not just floating in an overlying sea of pus. That today's sophisticated minimally invasive surgery does not directly treat the surface bacteria and fungi may be a significant reason for failures in sinus surgery. Simple washing at surgery even with topical antibiotics at high concentration and corticosteroids has not been shown to affect the success rate. Missing is a treatment directed specifically at the mucosa surface to kill bacteria and fungi at that micron thick level, within or below the biofilm that covers the cavity's surface.

If there is to be thorough cleaning and rehabilitation of the sinus mucosa, any biofilm on the surface needs to be removed. This must be done without destroying the delicate cilia or removing the eukaryotic or epithelial stem cells; otherwise the lining wall may become a scarred membrane incapable of producing or moving mucous and thus susceptible to repeat and chronic infection.

In several studies it has been shown that biofilms may be diminished or destroyed without any adverse effects on the eukaryotic cells by non-thermal plasmas. Non-thermal or cold plasma is produced as some type of electrical generator excites neighboring atoms and molecules to separate off electrons. The non-ionized gas remains at or very near ambient temperature because the heat is all in the very small electrons and larger mass radicals become barely warm. A gas, such as helium or argon, is excited into a plasma by passage between or near electrodes subjected to a voltage waveform that could be pulsed alternative or direct current to produce a plasma flow. The electrons and ions in the plasma flow are attracted to surfaces such as those within the human body. Electric fields generated and sustained in the plasma flow influence the biofilm and pathogen environment. The amount of current carried is miniscule and has no electrical effect since plasma is quasineutral. As the plasma degenerates the energy of the radicals and electrons is released as ultraviolet radiation. This latter form of energy is also active and effective. The radicals produced, termed reactive oxygen species (ROS), are creations of oxygen and nitrogen and water as found in air or other surrounding environment. Their chemical interactions with bacteria and tissue (plasma chemistry) are a prime means of effecting sterilization.

### SUMMARY OF THE INVENTION

The present invention is accordingly directed to methods and apparatus for treating biofilms on internal organs by the direct or indirect application of cold plasma and more particularly to the treatment of acute and chronic sinusitis through the use of a novel endoscope which may be passed into the sinus cavities and directs cold plasma or plasma-activated species (referred to as “active species”) to the infected mucosal surface.

In a preferred embodiment of the present invention, the non-thermal plasma (cold plasma) is supplied to the mucosal surfaces within the sinus cavities using an endoscope that a surgeon may insert through the patient's nasal cavity, and possibly through a small surgically created window in the sinus cavity.

The plasma may be generated by applying an RF alternating current of any frequency or a pulsed direct current to a flow of any useful gas, or gas mixture, such as noble plus oxygen. This may be done near the proximal end of the endoscope by applying the electrical power to spaced electrodes in the gas flow and allowing the gas flow to carry the resulting plasma to one or more outlets near the distal end of the endoscope, or by generating the plasma at the distal end either by the application of current or voltage between spaced electrodes or through a known technique of dielectric barrier discharge (DBD). In the following description and claims these plasma generation mechanisms may be collectively referred to as “cold plasma generators”.

The endoscope tube must be very narrow, preferably smaller than about 4.5 millimeters in diameter or smaller for use in the sinuses, while endoscopes for gastrointestinal use or the like may be longer. It includes a channel which is nonconductive if the flow plasma technique is used, and possibly additional channels for irrigation flow, suction and therapeutic agent delivery. It also includes optical fibers for illumination of the surgical site and for imaging the site from the proximal end through an optical fiber having a focusing lens at the distal end. At least the distal end of the tube is flexible, and a steering mechanism controlled by the surgeon at the proximal end is connected to the flexible section with wires or the like extending through the tube.

Alternatively, the distal end of the endoscope may be curved so that the end extends at an angle to the straight proximal section. The surgeon may then manipulate the distal end of the endoscope through body passages to reach the treatment area.

In another embodiment, a flexible casing enclosing the working channels may be disposable. In this case the disposable part is clipped into a metal hand piece. This metal hand piece may contain the fiberoptic viewing bundle and light carrier. In this case the distal end of the hand piece may be flexible thus bending the casing.

In another embodiment of the invention, in order to cut through the maxillary sinus, or other structures, or to coagulate bleeding, a quartz fiber may carry a beam from a proximal end laser through the tube.

In some embodiments, different gases and particular combinations of these gases, which may be varied by the surgeon during the operation, may be provided through use of a variety of gas sources at the proximal end of the endoscope, and surgeon controlled valves and mixers to selectively connect the gases to a channel. Certain of the gases and their combinations are optimized for generating plasmas which can cut tissues, destroy bacterial bodies or coagulate bleeding and they may be selectively used by the surgeon during a procedure. Control may be facilitated by feedback from current, voltage, temperature, pressure, and optical or other means.

In order to spread the application of a plasma over the sinus area to be treated, another embodiment utilizes a double balloon diffuser to receive gas at the distal end, feed it between the balloons, the inner having a conductive coating, such as aluminum on its inner surface.

This Mylar balloon with its electrode surface sits within an outer balloon. The volume between the balloons is filled with the pressurized gas through passages in the inner balloon. But in addition this outer balloon has multiple tiny holes and may take the form of mesh. Short fibrous webs connect the inner surface of the outer balloon and the outer surface of the inner balloon to maintain a uniform spacing between the two. Alternatively, a plastic foam could be dispensed between the balloons to maintain the spacing. The gas will be ionized as the outer surface of the balloon is brought into close proximity with a body surface, such as a mucous membrane which acts as a ground and the resulting plasma will fill the volume between the two balloons and extend through the mesh as multiple plumes which treat the body surface. A ground-like connector could alternatively be provided in other ways, such as by a conductor extending from the second terminal of the power supply to the gas in the vicinity of the balloon.

### BRIEF DESCRIPTION OF THE DRAWINGS

Other objects, advantages, and applications of the present invention will be made clear by the following detailed description of preferred embodiments. The description makes reference to the accompanying drawings, in which:

FIG. 1 is a perspective view of an endoscope formed in accordance with the preferred embodiment of the invention being used to operate on a patient;

FIG. 2 is a detailed distal end section of the endoscope of FIG. 1;

FIG. 3 is a cross-sectional view of a patient's head showing the path for the passage of the endoscope into the sinus area;

FIG. 4 is a perspective view of the distal end, broken away, of an embodiment of the invention employing dielectric barrier discharge to generate plasma;

FIG. 5 is an illustration of a conformal balloon used to expand into the contours of the sinus cavity and generate local plasma for therapeutic application;

FIG. 6 is a perspective view of a therapeutic endoscope with multiple channels showing an insertable cold plasma applicator; and

FIG. 7 is a perspective view of the insertable cold plasma applicator in a flexed position.

### DETAILED DESCRIPTION OF THE PREFERRED EMBODIMENTS

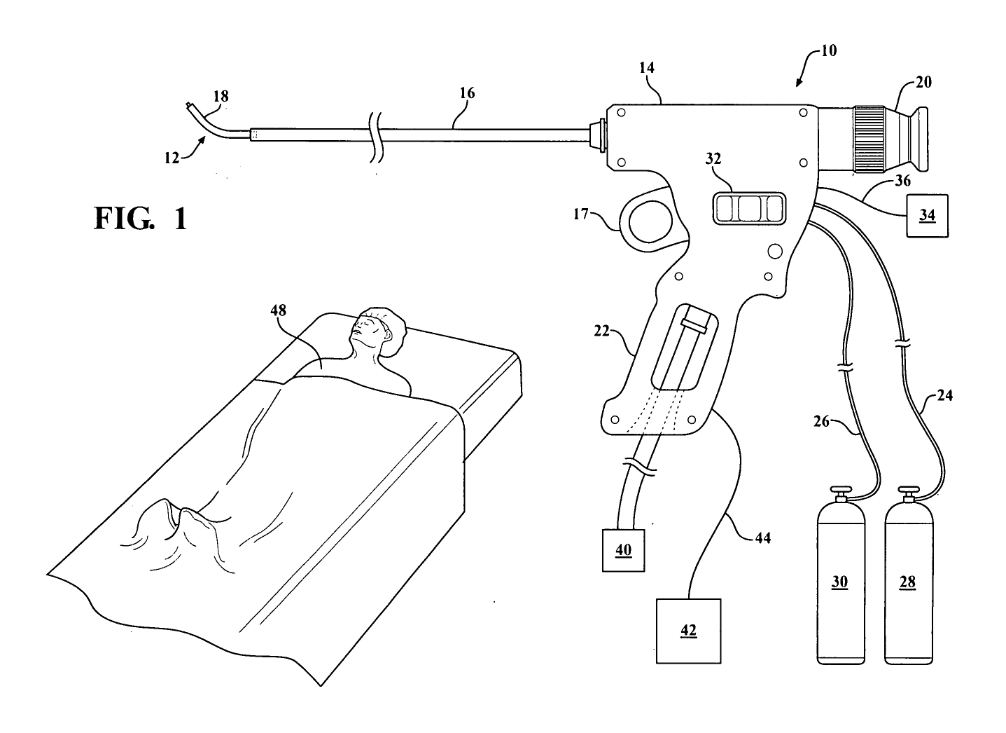

Referring to the drawings, FIG. 1 is a side view of a first embodiment of the invention, constituting an endoscope capable of cutting tissue using a laser and treating the tissue with a cold plasma tube to destroy biofilm, coagulate bleeding tissue, and sterilize the operative area. Preferably the system uses a non-thermal plasma operating into an atmospheric pressure using either direct or indirect plasma. Preferably the endoscope of the present invention comprises a non-disposable section generally indicated at 10 and a disposable distal section generally indicated at 12 which may be joined to the section 10 to form a unitary instrument.

The non-disposable section 10 generally comprises a hand-held section 14 having an extending rigid tube 16 projecting from it. The hand-held section includes a trigger grip 17 which may be pivoted by the surgeon to control the curvature of the distal tube 18 forming part of the disposable section 12. This is done to manipulate the distal section through tortuous passages such as nasal cavities surrounding sinuses. The section 14 is one of many designs for the proximal end of the device that controls movement, holds the flexible and steerable tubing/conduit, joins the various gas lines to the device and encloses the chamber to produce the plasma.

While the invention as disclosed is particularly useful for the treatment of sinusitis by the destruction of the biofilm covering of the mucosal lining of the sinuses which characterizes sinusitis it may be used in connection with other internal body cavities and organs.

The hand-held section also includes an eye piece 20 through which the surgeon may observe the body areas at the distal end of the tube 18. Alternatively, a small digital camera could be disposed at the eyepiece location to allow an enlarged display on a monitor. This is done through a conventional fiber-optic viewing system which may include an illumination source for the body areas at the distal end. The hand-held section 14 also includes a conventional gripping section 22 which the surgeon holds and uses to manipulate the distal end of the tube 18 into an operative position. While as illustrated the endoscope is only capable of bending the tube 18 in a single plane, by manual rotation of the hand-held section 14 through a plane transverse to the plane in which the tube section 18 may be bent, the distal end of the tube 18 may be moved in three dimensions. In other embodiments of the invention the trigger 17 may be replaced by a joystick type control that could manipulate the end section 18 in three dimensions. The invention could alternatively employ an endoscopic tube that does not have a flexible distal section, but is rather rigid with a straight tube extending from the proximal end to a curved but rigid distal end. The surgeon using this tube must rotate the endoscope as it passes through the body to follow curved passages and the like.

The hand-held section also receives flexible tubing members 24 and 26 which are connected, respectively, to two gas sources 28 and 30 which may be alternatively or collectively used to create the plasma. While two sources are illustrated, in other embodiments of the invention three or more gas supplies might be connected. Through use of the control 32 on the hand-held section 14 the surgeon may select one or more of the gas sources, or selected combinations of gases, for flow into the plasma. A preferred combination comprises a noble gas, such as argon, with a small percentage of oxygen to promote the generation of reactive oxygen species when the cold plasma is formed.

In many surgical procedures it may be necessary to perform more than a single operatory mode. For example, in the treatment of sinusitis it may be necessary to cut bone or soft tissue to gain access to a sinus space with an endoscope to reach a region of diseased tissue and then the diseased region must be treated by a separate instrument.

For example, operating on the biofilm formed in the mucosal lining of a sinus may be achieved by first connecting a gas source optimized for cutting bone so as to allow passage of the distal end of the plasma tube into the sinus cavity, then switching the gas source to a composition optimized for treating the biofilm, and finally again changing the plasma gas to a composition optimized for coagulating any blood flow or cauterizing damaged tissue or removing/vaporizing any residual obstructing tissue.

The gas connections may be made through surgeon adjustable valves, which may be opened, closed, or adjusted to an intermediate flow rate. This allows the composition and flow rate of the plasma forming gases to be adjusted to optimize the phase of the operation that the surgeon deems appropriate and to change those variables for each phase of the procedure.

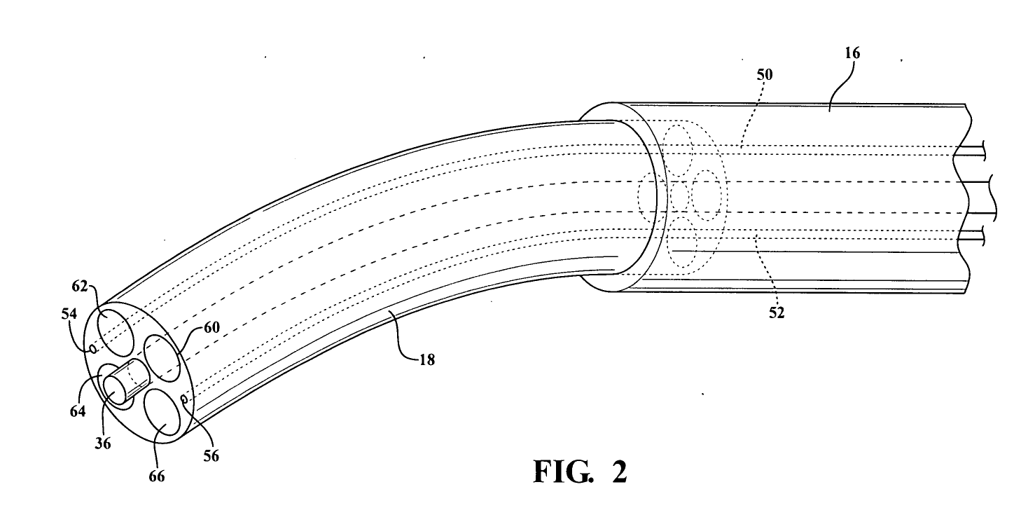

The hand-held member 14 also connects to a laser 34 through a fiber 36 which then passes through the output tube 16. The laser beam carried by the fiber 36 would be used to cut through any interfering structures necessary to bring the distal end into the operative area and the treatment would proceed with a plasma generated by the gas flowing through the tube 16. The laser beam could also be used to coagulate any bleeding tissues produced by the cutting and the cold plasma can also coagulate bleeding tissue. This structure could be incorporated into the endoscope of FIGS. 1 and 2 or could be a standalone endoscopic structure operative to cut, coagulate any blood produced, destroy any biofilm, and sanitize the area.

Another channel may connect a flow of liquid, possibly containing surfactants and/or biofilm reducing agents and/or antimicrobial agents and/or anti-inflammatory agents such as corticosteroids, from a source and sink 40. A power supply 42, preferably a RF source, has its output connected to the hand-held unit 14 by the conductor 44. Since there is net zero current flow to the patient 48 when the neutral plasma interacts with the patient, no grounding is necessary.

As shown in FIG. 2 a pair of guide wires 50 and 52 shown in phantom extend from the hand-held unit 14 and connect to anchor points 54 and 56 respectively on diametrically opposed points on the open end of the disposable tube 18. Forces imposed on the tube guide wires by motion of the trigger 17 cause the flexible end section 18 to bend relative to the primary tube 16. The entire length of the tube 16 and its flexible extension 18 may be in the range of 13 centimeters and the diameters of the tubes are preferably 4 millimeters or less. This allows for maneuvering through the nasal passages.

As shown in FIG. 2 four channels, 60, 62, 64 and 66 extend the entire length of the tubes 16 and 18. These channels are preferably part of the disposable section joined with complimentary channels which extend through the tube 16 and terminate at its end. In other embodiments of the invention there may be a different number of channels such as 1 to 6. One of the channels, such as 60, carries the optical fiber which allows the surgeon to view the operative area through the eye piece 20. The channel 62 may either carry the plasma generated within the hand-held section 14 by excitation of the gas entering there, which plasma then passes through the length of the tubes 62, or may carry a gas which is ignited by a suitable dielectric barrier discharge device (not shown) disposed at the distal end. Similarly channel 64 may carry a plasma generated by a different gas, with a different igniting voltage so that one of the two plasmas could be used for coagulation of the bleeding which occurs when the plasma is directed to the biofilm. Other means of igniting a plasma may be used such as induction.

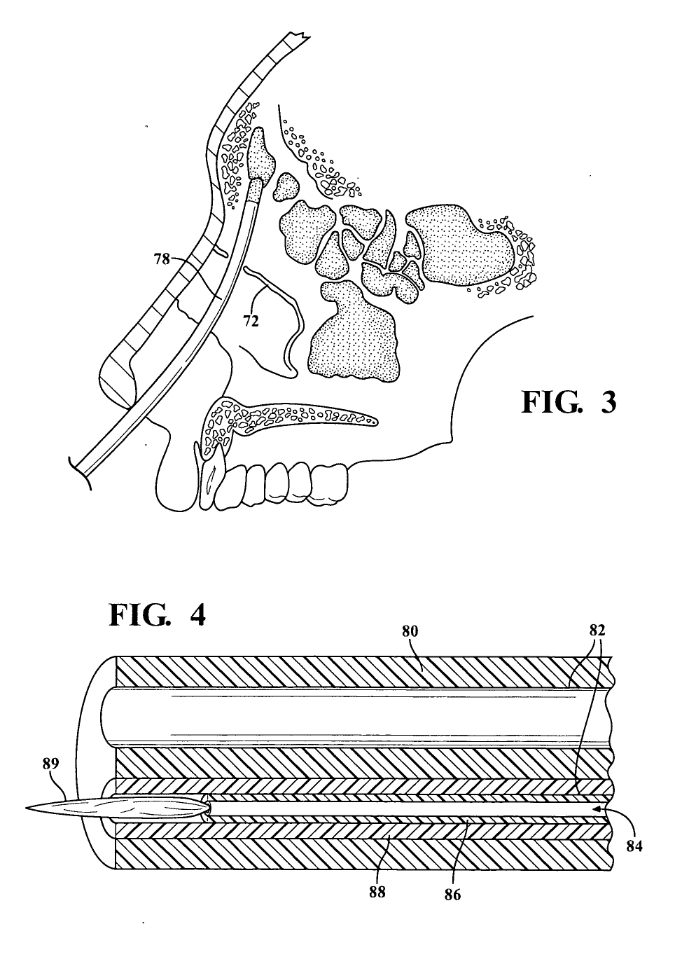

FIG. 3 is a cross section through a human skull at the nasal and sinus areas illustrating the tube 18 generating a plasma plume adjacent to a point on the mucosal lining which is coated with biofilm. The thin bone layer 72 surrounding the sinuses, which must be broken or cut by the laser to allow passage of tube 18 is shown in this figure. Already available balloon dilatation devices can be passed through the channel to expand a sinus ostium.

FIG. 4 illustrates the distal end of an embodiment of the invention in which the plasma is generated at the distal end by a dielectric barrier discharge (DBD) or other conventional technique to form a plasma jet. The endoscope 80 is illustrated as having two channels 82 and 84, the latter of which has grounded walls. A tubular high voltage RF or pulsed electrode 86, which terminates shortly before the channel 84, and has gas flowing through it and out the end, is disposed in the channel 84. The channel 84 has a dielectric tubular liner 88, which covers the electrode 86. The dielectric tubular liner 88 may cover only the outer surface of electrode 86, or it may cover all surfaces of electrode 86. A plasma stream 90 is generated at the end of the electrode 86 and flows out of the endoscope.

Since the device of FIG. 4 has a voltage waveform that is alternating above and below ground there is no net flowing current into the patient or worker, so grounding issues are mitigated. The dielectric barrier in the device inhibits arc formation and since it is low-power the thermal effects are minimal. The dielectric barrier thickness can be adjusted to achieve breakdown of the gas and creation of a plasma. High-permittivity bio-compatible materials, such as titanium dioxide or barium titanate, could be used to enhance surface electric fields and improve applicator performance. These materials allow the powered electrode tip to remain electrically insulated from arcing to the electrode while still providing high time-varying conduction via displacement current (capacitive action). This can be particularly important in small geometries, such as an endoscope channel, where the location of the electrodes carrying the high-voltage high-frequency drive are constrained. The use of high dielectric constant materials allows the high-voltage electrode to be placed far from the plasma region (well insulated) with little loss of coupling efficiency. This gives control over where the plasma is formed without the risk of arcing.

One problem in the plasma treatment of body surfaces in general, and internal organs in particular, when application time is limited, is that the area to be treated is relatively large compared to the dimensions of the plasma and the surface is often irregular. Both of these factors extend the treatment time by requiring the area to be scanned by a plasma plume on a point to point basis.

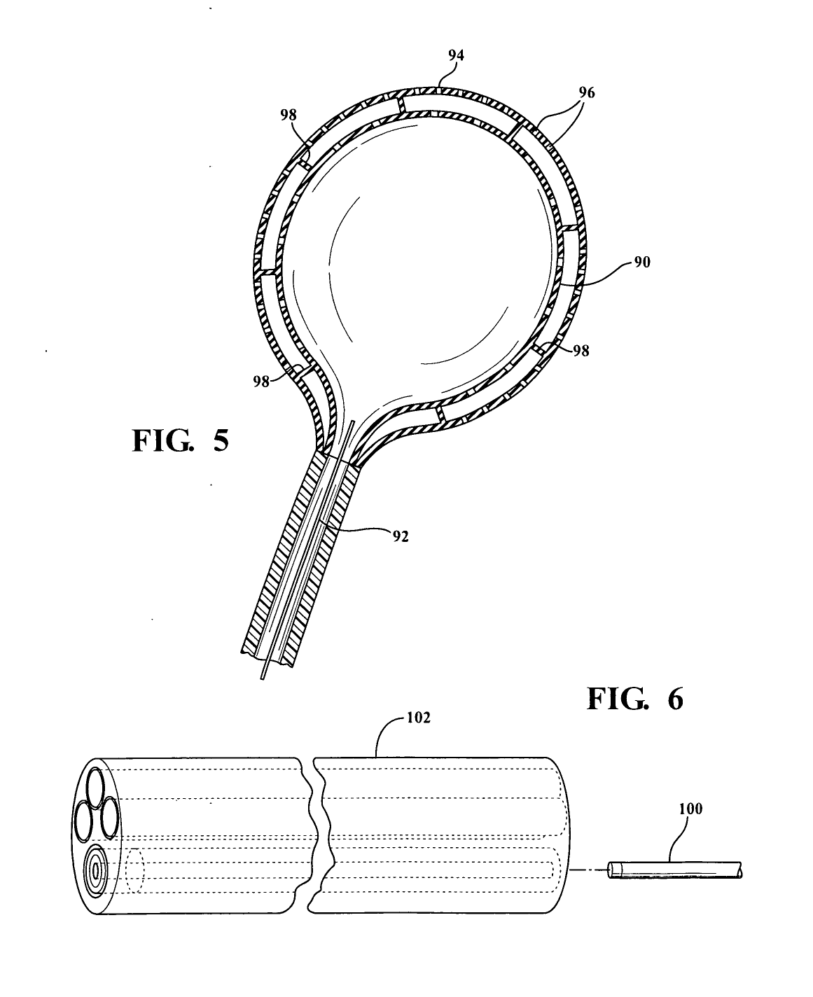

An embodiment of the invention illustrated in FIG. 5 addresses these problems by generating the plasma within a balloon structure which conforms to irregular surfaces and spreads the plasma over a relatively large area. The inner balloon 90 has a metalized coating on the inside and preferably is formed of Mylar. The metal coating is connected to one terminal of a high voltage RF power source 92 and the balloon is filled with pressurized gas. The effect of charging the conductive inner surface is to create charged particles which accumulate on the dielectric outer surface of this balloon.

This Mylar balloon 90 with its electrode surface sits within an outer nonconductive balloon 94. The volume between the balloons is filled with the pressurized gas through passages in the inner balloon. But in addition this outer balloon 94 has multiple tiny holes 96 over its surface and may take the form of mesh. Short fibrous webs 98, or plastic foam, or bubbles of a liquid connect the inner surface of the outer balloon 94 and the outer surface of the inner balloon 90 to maintain a uniform spacing between the two. The gas will be ionized as the outer surface of the balloon is brought into close proximity with a body surface, such as a mucous membrane which acts as a ground and the resulting plasma will fill the volume between the two balloons and extend through the mesh as multiple plumes which treat the body surface. The ground connector could alternatively be provided in other ways, such as by a conductor extending from the second terminal of the power supply to the gas in the vicinity of the balloon.

This double balloon arrangement can be applied to the mucous membrane close enough that effective sterilization can occur. Depending on the thickness of the balloons they can be deployed in a cavity to cover all regions of the surface so that with one application most if not all the mucous membrane is treated, as the balloons will deform to conform with the cavity surfaces. In some situations the balloon may inflate enough to flatten irregularities of the surface and assure plasma presentation to all surfaces.

In a basic preferred configuration a more or less spherical balloon can be inflated to fit snuggly in the maxillary and/or sphenoid sinuses. A variation of this would be a cylindrical shape that would fill the ethmoid sinuses, or a pyramidal shape that would fill the frontal sinuses.

This same preformed shaping of the balloon would be used to treat enclosed spaces, for example but not limited to: the urinary bladder, the ureter or urethra, the tracheal bronchial tree, orthopedic joints, the gastrointestinal track including the cystic and pancreatic ducts as well as having an effect on blood vessels and heart valves.

Another preferred application would be to treat bleeding in an area such as the nose where there are rigid submucosal structures (bone and cartilage). The balloon that is treating the nosebleed can be inflated with enough pressure to stop the bleeding by pressure and the plasma can cause coagulation.

In an alternative embodiment of the invention, the plasma generator takes the form of an attachment to a conventional endoscope. Therapeutic functional endoscopes on the market have flexible small-diameter instrumentation channels that normally would carry suction, irrigation or forceps (e.g. Olympus ENF-VT2/T3), as well as an open channel which may be used by the surgeon to insert various medical instruments or manipulators. The present invention contemplates insertable plasma applicators that can be added to any therapeutic endoscopic device or small diameter integrated endoscopes for the paranasal sinuses, where space is a premium. These insertable devices should have a diameter of less than about 2.5 millimeters.

Inserts formed in accordance with the present invention may be inserted into one of these open channels to create an instrument for generating a cold plasma plume at the distal end of the endoscope for the treatment of organs.

These inserts may generate a plasma at the proximal end that is carried by the pressure of flowing gas out the distal end. Alternatively, the plasma may be generated at the distal end by passing gas and an electrical conductor which is connected to an RF source or the like at the proximal end, through an open channel in the endoscope.

When the flowing plasma insert is used, the flow channel is preferably nonconductive to prevent dissipation of the plasma, and if the endoscope channel is formed of conductive material, a nonconductive tube is required as part of the insert.

The insert is preferably disposable, although the electronics associated with the RF generator may be preserved. Such RF electronics, gas manifold, and control hardware could be located at the proximal end of the endoscope for easy user adjustment.

The plasma generator shown in FIG. 4 represents the distal end of an endoscope and the gas flow tube and electrode may be of an insertable design. FIG. 6 illustrates the manner of insertion of a DBD plasma generator 100, like that of FIG. 4, into an endoscope 102, from the proximal end, in accordance with the present invention, but the generator can alternatively be inserted from the distal end.

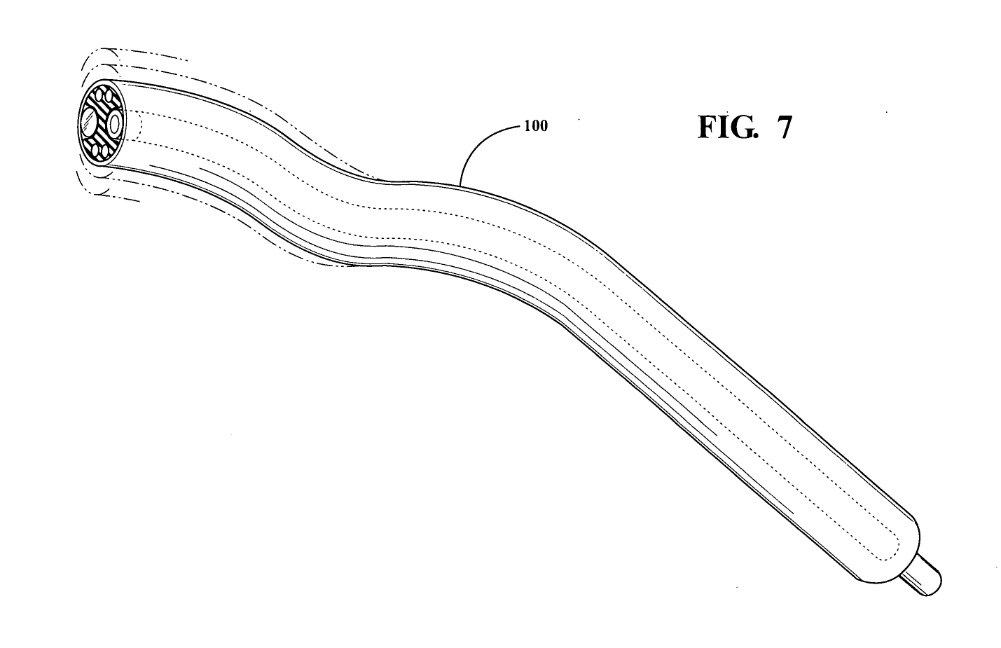

FIG. 7 illustrates the flexible nature of the plasma generating insert 100. This allows the distal end to flex when the insert is used in an endoscope with a flexible, bendable distal section, such as the endoscope 10. The flexible insert could also be used with an endoscope without a bendable section but rather a curved distal section which the surgeon must rotate to navigate through body passages. This allows the insert, which may extend beyond the distal end of the rigid tube with the bent end, to be disposable. The bend end section of the rigid outer tube may also be removable and disposable.

Having thus described our invention, we claim:

1. An endoscope for the treatment of internal body organs affected by pathogens, to reduce the pathogens, comprising:
an elongated tube adapted to be inserted into a body so that its proximal end is external of the body and its distal end is proximate said body organ surface;
an electrical power source;
a source of gases;
a cold plasma generator connected to said electrical power source and said source of gases to generate cold plasma; and
at least one channel in the endoscope to carry activated species created by said plasma source to the surface to be treated.

2. The endoscope for the treatment of internal body organs of claim 1 wherein the source of gases comprises a plurality of containers of pressurized gases and valves connecting the containers to said channels selectively, or as a combined mixture.

3. The endoscope of claim 1 wherein the plasma and plasma-activated species is created adjacent to the proximal end of the tube so that the plasma-activated species flows by gas pressure through the tube to the distal end.

4. The endoscope of claim 1 wherein the plasma is created at the distal end of the tube and the pressurized gas and the output of the power source extends from the proximal end of the tube to the plasma creation zone at the distal end of the tube.

5. The endoscope of claim 1 wherein the cold plasma generator used to create the cold plasma is a dielectric barrier discharge device.

6. The endoscope of claim 5 where the dielectric barrier thickness can be adjusted to achieve breakdown of the gas and creation of a plasma.

7. The endoscope of claim 5 where a high-permittivity bio-compatible material is used as at least a portion of the dielectric barrier.

8. The endoscope of claim 1 wherein the power source constitutes a radio frequency generator.

9. The endoscope of claim 1 wherein the power source outputs a pulsed signal.

10. The endoscope of claim 1 wherein at least a portion of said tube is flexible and further comprising a manually operated control member at the proximal end of the tube connected to the flexible section to allow a user to control the curvature of the flexible section for manipulation of the tube within a body.

11. The endoscope of claim 10 wherein the manually operable control member at the proximal end of the tube is connected to the flexible section of the tube to control the bending of the tube through control wires.

12. The endoscope of claim 1 further comprising a first fiberoptic bundle extending through the endoscope tube and connecting to said light source at the proximal end and a second fiberoptic bundle extending through the endoscope connecting to a focusing lens at the distal end and an eyepiece at the proximal end.

13. The endoscope of claim 1 wherein said channels extending through the length of the endoscope provide for the flow of irrigation fluids, suction forces, and the like.

14. An endoscope to be inserted into a body to apply active species to the surfaces of internal organs to reduce pathogens on said surfaces, comprising:
an elongated cylindrical tube having a proximal end and a distal end adapted to be positioned adjacent to the organ surfaces;
the proximal end of the tube being rigid with a relatively flexible section of the tube adjacent the distal end;
control wires extending through the tube and terminating at the flexible section;
a manually operable control member at the proximal end of the tube connected to the proximal ends of the wires to allow a user to control the curvature of the flexible section for manipulation of the tube within a body;
a source of gases;
a electrical power source; and
a cold plasma generator connected to said source of gases and electrical power source and operative to pass activated species through the distal end to said organ surfaces.

15. The endoscope of claim 14 further comprising a laser at the proximal end, and a quartz fiber extending through the length of the tube for carrying a beam generated by the laser to an output at the distal end for projection on to said internal organ surfaces.

16. The method of treating surfaces of internal organs in a human to reduce pathogens on the surface of said organs comprising:
inserting an endoscope comprising an elongated tube having a distal end and a proximal end into a body cavity to bring the distal end into proximity to the surface of the internal organ to be treated while the proximal end is external of the body;
connecting the proximal end of the endoscope to a source of gas and to an electrical power source;
using the power source to excite the gas to create a cold plasma; and
applying the resulting activated species to the surfaces to be treated through the endoscope tube.

17. The method of claim 16 wherein the internal organ surface to be treated constitutes the mucosal lining of a nasal sinus.

18. The method of treatment of surfaces of internal organs of claim 16 further comprising generating the cold plasma at the proximal end of the endoscope tube and passing a stream of cold plasma through the tube and out the distal end to apply the resulting activated species to the internal organ.

19. The method of claim 16 in which electrical conductors carrying the electrical power and the gases pass from the proximal end of the endoscope to the distal end, where the power source turns the gas into a cold plasma, generating activated species which are applied to the organ surfaces to be treated.

20. The method of claim 16 in which the power source is used to turn the gas flow into a cold plasma employing a dielectric barrier discharge technique.

21. The method claim 16 in which the gas comprises a noble gas and oxygen.

22. An endoscope for the treatment of internal body organs having pathogens on their surfaces comprising:
an elongated cylindrical tube having a proximal end and a distal end, the distal end being adapted to be inserted into the body in proximity to said body organs to be treated with the proximal end external of the body;
a source of gas;
an electrical power source;
a power source to create a cold plasma;
a pair of balloons supported at the distal end of the endoscope tube and arranged with an inner balloon interior of an outer balloon;
a connection for passage of the gas through the tube to the proximal end of the tube so that the gases flow through the tube and into the interior of the interior balloon;
the interior balloon having a metalized coating on its interior side and apertures which allow pressurized gases to pass through said apertures into the space between the balloons;
the outer balloon having a number of apertures; and
a connection from the electrical power source extending into contact with the metalized coating on the inner balloon, whereby cold plasma will be generated in the volume between the two balloons and activated species flow out of the apertures in the outer balloon into contact with the body organs to be treated.

23. The endoscope of claim 22 wherein the gases comprise a noble gas.

24. The endoscope of claim 22 wherein the gases further comprise oxygen, nitrogen or water vapor.

25. A disposable attachment for an endoscope having a cylindrical channel with an inner diameter extending from the proximal end to the distal end, the attachment enabling the endoscope to apply plasma-activated species to the surface of internal organs to reduce pathogens on said surfaces, comprising:
a flexible, elongated cylindrical tube having an outer diameter smaller than the inner diameter of said channel, enabling the tube to be inserted into the channel;
a source of gases adapted to be connected to the interior of the tube to allow the passage of gases through the tube;
a power source to create the activated species.

26. The attachment of claim 25 where the attachment is adapted to be placed in an endoscope containing fiberoptic bundles to illuminate and visualize the operative site.

27. The attachment of claim 25 where the attachment is adapted to be bent at the free distal end by control wires.
